(19) United States
(12) Patent Application Publication
(10) Pub. No.: US 2026/0338752 A1
(43) Pub. Date: Oct. 29, 2026

(54) BINARY GAS ANALYZER

(71) Applicant: FuelCell Energy, Inc., Danbury, CT (US)

(72) Inventor: Pascal Tessier, Ridgefield, CT (US)

(21) Appl. No.: 19/568,413

(22) Filed: Mar. 16, 2026

Related U.S. Application Data
(60) Provisional application No. 63/793,050, filed on Apr. 23, 2025.

(51) Int. Cl. G01N 33/00; G01N 29/024; G01N 29/44

(52) U.S. Cl. CPC G01N 33/005; G01N 29/024; G01N 29/4472

(57) ABSTRACT

A hydrogen production system including an electrolysis cell system with a fuel electrode portion configured to output a fuel electrode output stream consisting substantially of hydrogen, nitrogen, argon, and water vapor and an air electrode portion configured to receive an air electrode input stream comprising air, a drying system configured to dry the fuel electrode output stream, and a gas analyzer configured to receive a first portion of the dried fuel electrode output stream. The analyzer includes a sound generator, a sound sensor, and a controller. The controller is configured to cause the sound generator to generate a sound, to determine the speed of a sound through the first portion based on a signal from the sensor, and to calculate the concentration of hydrogen in the first portion based on the speed of the sound and assuming the concentration of argon is approximately 1/78 the concentration of nitrogen.

### CROSS-REFERENCE TO RELATED PATENT APPLICATIONS

This Application claims the benefit of and priority to U.S. Provisional Patent Application No. 63/793,050, filed Apr. 23, 2025, which is incorporated by reference herein in its entirety.

### BACKGROUND

The present disclosure relates generally to the field of gas production and analysis. In particular, the present disclosure relates to the analysis of hydrogen and oxygen produced by electrolysis.

The speed of a sound through a gas mixture can be determined using the formula:

$$S ( mix ) = \sqrt{\frac{{γ}_{mix} RT}{{M}_{mix}}}$$

where

$${M}_{mix} = \sum {c}_{i} {M}_{i} , {γ}_{mix} = 1 + \frac{R}{\sum {c}_{i} {M}_{i} {C}_{{V}_{i}}} ,$$

T is the temperature of the gas, M is the molar mass of a particular gas species, c is the percentage of a particular gas species in the mixture, R is the ideal gas constant, and C_{V} is the specific heat of a particular gas species.

When a gas mixture contains only two gases, the percentage or concentration of a first gas and the percentage of the second gas add up to 100%. Thus, the concentration of the second gas species c₂ can be defined as 100%−c₁ (or 1−c₁). By replacing c₂ with 1−c₁ in the formulas above and measuring the speed of a sound through the mixture, c₁ becomes the only unknown and can easily be determined. c₂ can then be calculated using the formula 1−c₁. Accordingly, when a gas mixture includes only two gas species, the percentage (or concentration) of each gas may be determined by measuring the speed of a sound through the mixture. However, when a third gas species is present in the mixture, the concentrations of the gas species cannot be expressed as a function of the concentration of only one of the other gas species. For example, the concentration of a third gas species c₃ may be expressed as 1−(c₁+c₂), but if c₃ were replaced with this formula, the formulas above would still have two unknowns and would not be solvable only by measuring the speed of a sound through the gas mixture.

Electrolysis cells are electrochemical cells configured to use an electrical current to split water vapor molecules into hydrogen and oxygen. In a solid oxide electrolysis system, water vapor is provided to a fuel electrode of the electrolysis cell, where the water vapor is split into hydrogen gas and oxide ions. The oxide ions cross an electrolyte to an air electrode. An air stream is provided to the air electrode as a sweep gas to dilute the oxygen and carry it out of the cell. Some of the nitrogen, oxygen, and argon in the air may cross over the electrolyte through cracks and leaks in the cell to the fuel electrode. The fuel electrode may thus output an exhaust stream including hydrogen generated in the electrolysis reactions, water vapor formed by the reaction of oxygen that crosses over from the air electrode (rather than as oxide ions through the electrolyte) with hydrogen in the fuel electrode, and nitrogen and argon that cross over from the air electrode. Water vapor that passes through the cell without being electrolyzed can also be present, although this is not a concern when dry air is used to sweep the air side of the solid oxide electrolysis system. Binary gas analyzers for determining the concentrations of gas species in a binary gas mixture (e.g., with only two gas species) may typically not be suitable for determining the concentrations of the gas species in the fuel electrode exhaust stream, which may include at least four gas species. However, these binary gas analyzers are much less expensive than other gas analyzers used to measure the concentrations of gases in mixtures with more than two gas species. Accordingly, it may be desirable to be able to use a binary gas analyzer to measure the concentrations of gas species in fuel electrode exhaust having more than two gas species.

### SUMMARY

Some aspects of the present disclosure relate to a hydrogen production system including an electrolysis cell system, a drying system, and a gas analyzer. The electrolysis cell system includes a fuel electrode portion configured to receive a fuel electrode input stream including gaseous fuel and to output a fuel electrode output stream consisting substantially of hydrogen, nitrogen, argon, and water vapor, and an air electrode portion configured to receive an air electrode input stream including air. The drying system is configured to dry the fuel electrode output stream. The gas analyzer is configured to receive a first portion of the dried fuel electrode output stream and includes a sound generator, a sound sensor, and a controller, the controller including at least one processor and at least one memory storing instructions that, when executed by the at least one processor, cause the controller to cause the sound generator to generate a sound, determine, based on a signal from the sound sensor, the speed of the sound through the first portion of the dried fuel electrode output stream, and calculate the concentration of hydrogen in the first portion of the dried fuel electrode output stream as a function of the speed of the sound through the first portion of the dried fuel electrode output stream, wherein the concentration of argon is assumed to be approximately 1/78 the concentration of nitrogen.

Other aspects of the present disclosure relate to a method of measuring the concentration of hydrogen in a gas mixture, the method including supplying a steam stream to a fuel electrode portion of an electrolysis cell system, supplying an air stream to an air electrode portion of the electrolysis cell system, drying a fuel electrode output stream output from the fuel electrode portion, the fuel electrode output stream consisting substantially of hydrogen, nitrogen, argon, and water vapor, measuring the speed of a sound in a first portion of the dried fuel electrode output stream; and calculating the concentration of hydrogen in the first portion of the dried fuel electrode output stream as a function of the speed of the sound through the first portion of the dried fuel electrode output stream, wherein the concentration of argon is assumed to be approximately 1/78 the concentration of nitrogen.

Other aspects of the present disclosure relate to a gas analyzer including a detection chamber, a sound generator positioned on a first side of the detection chamber; a sound sensor positioned on a second side of the detection chamber, and a controller including at least one processor and at least one memory storing instructions that, when executed by the at least one processor, cause the controller to cause the sound generator to generate a sound, determine, based on a signal from the sound sensor, the speed of the sound through a gas mixture in the detection chamber, the gas mixture consisting substantially of hydrogen, nitrogen, and argon, or hydrogen, nitrogen, argon, and water vapor, and calculate the concentration of hydrogen in the gas mixture as a function of the speed of the sound through the gas mixture, wherein the concentration of argon is assumed to be approximately 1/78 the concentration of nitrogen.

Other aspects of the present disclosure relate to a method of analyzing the contents of a gas mixture consisting substantially of a condensable vapor, a first gas species, a second gas species, and a third gas species, the second gas species and the third gas species being present in the gas mixture in a known ratio. The method includes condensing the vapor to form a liquid and separating the liquid from the residual gas mixture, measuring the speed of a sound through the residual gas mixture, and calculating the concentration of the first gas species in the residual gas mixture as a function of the speed of the sound through the residual gas mixture, wherein the concentrations of the second gas species and the third gas species are presumed to be in the known ratio.

Other aspects of the present disclosure relate to a method of analyzing the contents of a gas mixture consisting substantially of a condensable vapor, a first gas species, a second gas species, and a third gas species, the second gas species and the third gas species being present in the gas mixture in a known ratio. The method includes condensing the vapor to form a liquid and separating the liquid from the residual gas mixture supplying the residual gas mixture to a binary gas analyzer configured to measure the speed of a sound through the residual gas mixture receiving, from the binary gas analyzer, an estimated concentration of the first gas species and an estimated concentration of the second gas species, determining, based on the estimated concentration of the first gas species and the estimated concentration of the second gas species, the speed of the sound through the gas mixture; and calculating the concentration of the first gas species in the residual gas mixture as a function of the speed of the sound through the residual gas mixture, wherein the concentrations of the second gas species and the third gas species are presumed to be in the known ratio.

### BRIEF DESCRIPTION OF THE DRAWINGS

FIG. 1 is a schematic diagram of a solid oxide electrolysis cell, according to an exemplary embodiment.

FIGS. 2-4 are schematic diagrams of solid oxide electrolysis cell systems, according to exemplary embodiments.

FIG. 5 is a schematic diagram of a membrane dryer according to an exemplary embodiment.

FIG. 6 is a schematic diagram of a gas analyzer according to an exemplary embodiment.

FIG. 7 is a schematic diagram of solid oxide electrolysis cell systems, according to an exemplary embodiment.

FIG. 8 illustrates a method of measuring the concentration of hydrogen in a gas mixture produced by an electrolysis cell assembly, according to an exemplary embodiment.

It will be recognized that the Figures are schematic representations for purposes of illustration. The Figures are provided for the purpose of illustrating one or more implementations with the explicit understanding that the Figures will not be used to limit the scope of the meaning of the claims.

### DETAILED DESCRIPTION

The embodiments described herein relate to systems and methods for using a binary gas analyzer to determine the composition of a gas mixture containing more than two gas species. In particular, the systems and methods described herein may be used to determine the concentration of hydrogen in a fuel electrode exhaust stream produced by a solid oxide electrolysis cell (SOEC) system. Upstream process controls and the solid oxide electrolysis process may ensure that the only gases in the anode exhaust stream in significant concentrations are hydrogen, nitrogen, argon, and water vapor. In some cases, the water vapor may be eliminated in a drying process, for example, using a membrane dryer. The speed of a sound through the gas mixture is measured by the binary gas analyzer, and the concentration of hydrogen is determined as a function of the speed of the sound. Because it is substantially certain that the nitrogen and argon in the drying system output stream are present due to air crossover from the air electrode, it may be substantially certain that the nitrogen and argon present in substantially the same ratio as they are present in air, about 78:1 nitrogen to argon. By assuming that the concentration of argon is about 1/78 the concentration of nitrogen, the measurement from a binary gas analyzer may be used to determine the concentration of hydrogen in a mixture containing hydrogen, nitrogen, and argon. In some cases, water vapor may not be entirely eliminated, the concentration of water vapor may be measured separately to determine the concentration of water vapor, and the measurement from the binary gas analyzer may be used to determine the concentration of hydrogen in a gas mixture containing hydrogen, nitrogen, argon, and water vapor.

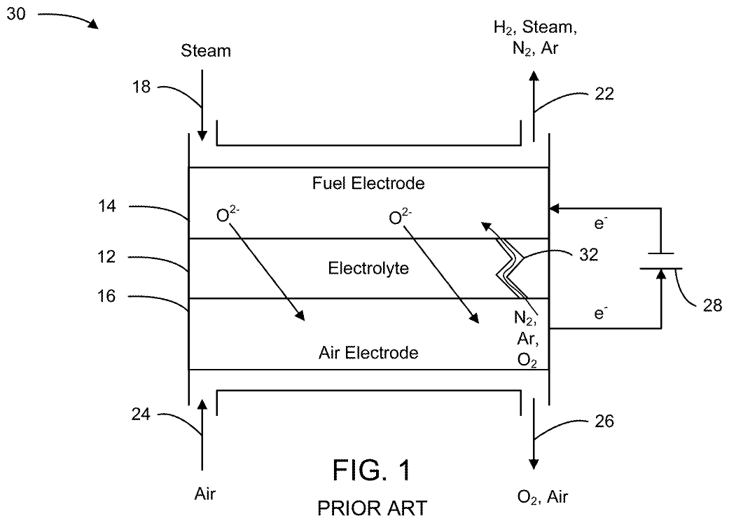

Referring to FIG. 1, an SOEC 30 is shown. The SOEC 30 includes an electrolyte 12 sandwiched between a fuel electrode 14 and an air electrode 16. A fuel electrode input stream 18 including water vapor is supplied to the fuel electrode 14. In some embodiments, water vapor supplied to the fuel electrode 14 of the SOEC 30 may be referred to as fuel. A power supply 28 supplies an electrical current to the SOEC 30, causing the water vapor to react at the fuel electrode 14 to form hydrogen gas and oxide ions. The fuel electrode 14 outputs a fuel electrode output stream 22 including the hydrogen gas and unreacted water vapor from the fuel electrode input stream 18. The oxide ions cross over the electrolyte 12 to the air electrode 16 where they react to form oxygen gas. The air electrode input stream 24 including air is supplied to the air electrode 16 to dilute the oxygen gas and sweep the oxygen gas out of the air electrode 16. The air electrode 16 outputs an air electrode output stream 26 including a mixture of the air and the oxygen gas. Nitrogen, oxygen, argon, and other atmospheric gases in trace amounts such as carbon dioxide in the air electrode input stream 24 may cross over to the fuel electrode 14 through cracks 32 in the electrolyte 12, gaps in seals between the fuel electrode 14 and the air electrode 16, and other potential pathways for crossover. At the operating temperature of the SOEC 30, oxygen leaking into the fuel electrode 14 readily reacts with hydrogen from the fuel electrode input stream 18, contributing a small amount of additional steam to the fuel electrode output stream 22. Thus, in addition to hydrogen and water vapor, the fuel electrode output stream 22 may include nitrogen, argon, and insignificant amounts of other atmospheric gases.

Multiple SOECs may be arranged in SOEC stacks, and multiple SOEC stacks may be arranged in an SOEC module. The SOEC module may include a module housing, which may act as a pressure vessel to maintain the SOEC stacks at an elevated pressure (e.g., above atmospheric pressure). The air electrodes of the SOECs in a module and the gas flow structures that move reactants and products into and out of the air electrodes may be collectively referred to as an air electrode section or portion or an anode section or portion. Similarly, the fuel electrodes of the SOECs in a module and the gas flow structures that move reactants and products into and out of the fuel electrodes may be collectively referred to as a fuel electrode section or portion or a cathode section or portion. In some cases, leaks may form between the fuel electrode portion and the air electrode portion, for example, if a seal between a gas flow passage in the fuel electrode portion and a gas flow passage in the air electrode portion fails, reactants may cross over into the opposite portion. For example, nitrogen, oxygen, and argon may leak into the fuel electrode portion from the air electrode portion. As discussed above, the oxygen may immediately react with hydrogen in the fuel electrode 14 to form additional water vapor at the typical operating temperature range of SOFC's and SOEC's. Thus, the exhaust stream from the fuel portion may consist substantially of hydrogen, water vapor, nitrogen, and argon. A binary gas analyzer typically could not be used to measure the hydrogen purity of the exhaust stream because the exhaust stream contains more than two gas species. However, the systems and methods disclosed herein allow for the use of a binary gas analyzer in an electrolysis cell system and accounting for additional impurities (i.e., more than one non-hydrogen gas species). These systems may reduce the cost of hydrogen purity analysis in electrolysis systems compared to other devices for measuring hydrogen purity in gas mixtures with more than two species, such as gas chromatography-based (GC) or Fourier transform infrared (FTIR) analyzers.

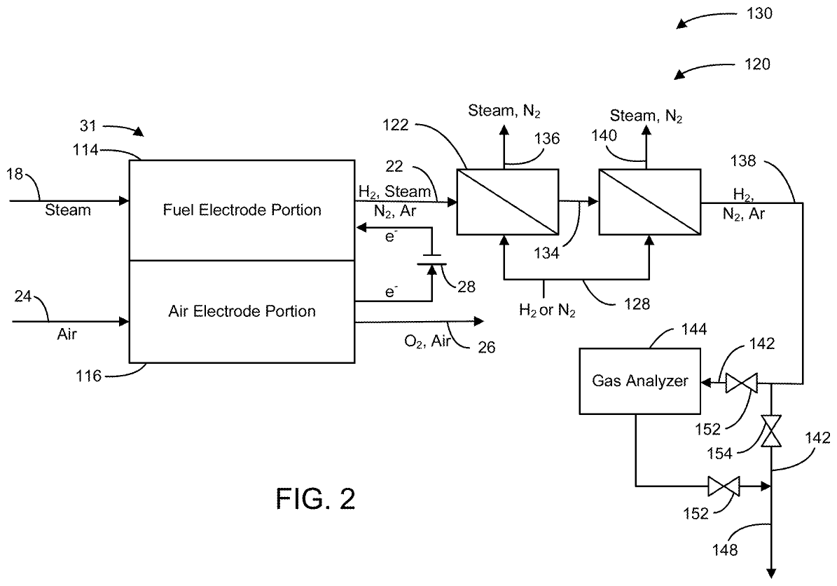

FIGS. 2-4 show three exemplary embodiments of an SOEC system 130 each with a different drying system 120. Referring now to FIG. 2, an SOEC system 130 is shown, according to an exemplary embodiment. The SOEC system 130 includes an SOEC assembly 31, which may be substantially similar to the SOEC 30 except that the SOEC assembly 31 may include multiple SOECs 30. For example, the SOEC assembly 31 may be an SOEC stack or SOEC module as discussed above. The SOEC assembly 31 includes a fuel electrode portion 114 representing one or more fuel electrodes and an air electrode portion 116 representing one or more air electrodes. The fuel electrode input stream 18 is supplied to the fuel electrodes in the fuel electrode portion 114, and the air electrode input stream 24 is supplied to the air electrodes in the air electrode portion 116. The fuel electrode portion 114 outputs the fuel electrode output stream 22 including the fuel electrode output from each of the fuel cells in the SOEC assembly 31, and the air electrode portion 116 outputs the fuel electrode output stream 22 including the air electrode output from each of the electrolysis cells in the SOEC assembly 31. Thus, the fuel electrode input stream 18 may include water vapor; the air electrode input stream 24 may include air; the fuel electrode output stream 22 may include hydrogen generated in the electrolysis reactions, water vapor that passes through the fuel electrode portion 114 without reacting, and nitrogen and argon that leaks through the electrolytes of the fuel cells; and the air electrode output stream 26 may include oxygen generated in the electrolysis reactions and air that passes through the air electrode portion 116 without reacting.

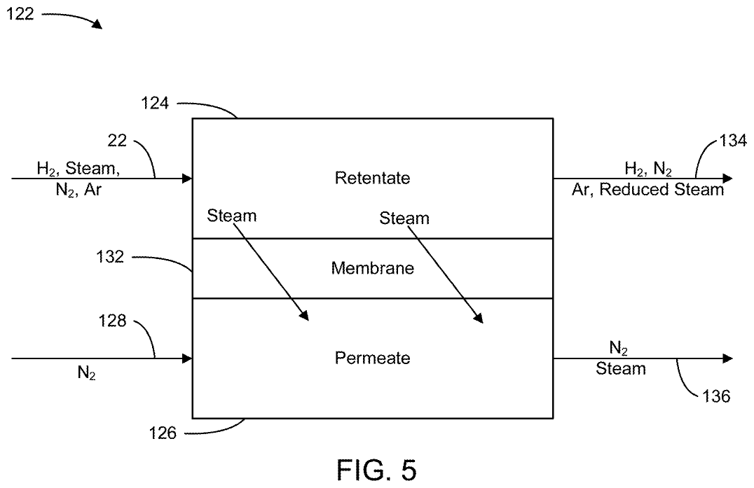

The SOEC system 130 of FIG. 2 includes a drying system 120 including two membrane dryers 122 in series. The membrane dryer 122 is shown in further detail in FIG. 5. The first membrane dryer 122 includes a retentate portion 124 configured to receive the fuel electrode output stream 22 and a permeate portion 126 configured to receive a sweep gas 128. The retentate portion 124 and the permeate portion 126 are separated by a membrane 132, which may be a water vapor-selective membrane (e.g., a polymer-based membrane) that preferably allows water vapor to pass therethrough. Water vapor in the retentate portion 124 may thus cross over the membrane 132 into the permeate portion 126. The sweep gas 128 sweeps the permeated water vapor out of the permeate portion 126, reducing the humidity and pressure in the permeate portion 126, which may allow more water vapor to cross the membrane 132. The retentate portion 124 may output a first retentate output stream 134 with less water vapor than the fuel electrode output stream 22 (e.g., zero water vapor), and the permeate portion 126 may output a first permeate output stream 136 including a mixture of water vapor and sweep gas.

Because a small amount of the sweep gas 128 may cross over the membrane 132 to the retentate portion 124, the sweep gas 128 may be hydrogen or nitrogen. Using hydrogen ensures that the hydrogen gas in the retentate portion 124 is not further diluted by sweep gas crossing over to the retentate portion 124. Using nitrogen may further dilute the hydrogen but will not introduce an additional contaminant not already present in the first retentate output stream 134.

To further dry the first retentate output stream 134, the first retentate output stream 134 is received by a retentate portion 124 of the second membrane dryer 122. Like the first membrane dryer 122, the second membrane dryer includes a permeate portion 126 that receives a sweep gas 128 and a membrane 132 separating the retentate portion 124 and the permeate portion 126. The retentate portion 124 may output a drying system output stream 138 with less water vapor than first retentate output stream 134, and the permeate portion 126 may output a second permeate output stream 140 including a mixture of water vapor and sweep gas. The drying system output stream 138 may also be referred to as the dried fuel electrode output stream. The second permeate output stream 140 may be substantially free of water vapor, which may simplify the determination of the hydrogen concentration using a binary gas analyzer. In some embodiments, the drying system 120 may have more than two membrane dryers 122. In some embodiments, the drying system may have only one membrane dryer 122.

A first portion 142 of the drying system output stream 138 may be supplied to the gas analyzer 144. The gas analyzer 144 may be referred to as a binary gas analyzer, as it measures the speed of a sound through a gas mixture to determine concentrations of gas species in a mixture, similar to typical binary gas analyzers. However, using the methods disclosed herein, the gas analyzer 144 can determine the concentrations of gases in a mixture containing more than two gas species. A second portion 146 containing the remainder of the drying system output stream 138 may bypass the binary gas analyzer 144, as only a relatively small sample of gas is needed to determine the hydrogen concentration of the drying system output stream 138. After the hydrogen concentration of the first portion 142 of the drying system output stream 138 is measured in the binary gas analyzer 144, the first portion 142 is recombined with the second portion 146 and output as a product hydrogen stream 148. The product hydrogen stream 148 may be sent to storage or used in various industrial processes. For example, the product hydrogen stream 148 may be compressed and stored in one or more storage tanks. The hydrogen concentration measured in the binary gas analyzer 144 may be associated with the respective storage tanks (e.g., in a computer database) and/or the storage tanks may be labeled with the measured hydrogen concentration.

The SOEC system 130 includes two shutoff valves 152, which can be closed to allow all of the drying system output stream 138 to bypass the binary gas analyzer 144. The SOEC system 130 further includes a pressure control valve 154 in the second portion 146 downstream of the location at which the first portion 142 splits from the second portion 146 and upstream of the location at which the first portion 142 is recombined with the second portion 146. When the shutoff valves 152 are open, the pressure control valve 154 may be adjusted to control the amount of the drying system output stream 138 is sent to the binary gas analyzer 144. For example, by partially closing the pressure control valve 154, more gas may flow to the binary gas analyzer 144 as pressure builds upstream of the pressure control valve 154. The opening of the pressure control valve 154 may be set such that an amount of gas needed for the hydrogen concentration measurement is sent to the binary gas analyzer 144.

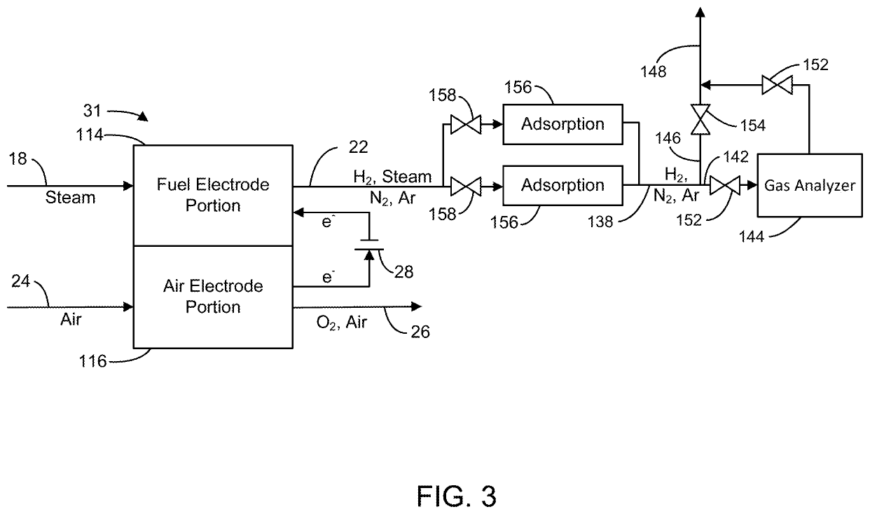

Referring now to FIG. 3, the SOEC system 130 is shown according to another exemplary embodiment. The SOEC system 130 of FIG. 3 may be substantially the same as the SOEC system 130 of FIG. 2 with the exception of the drying system 120. In the drying system 120 of FIG. 3, the fuel electrode output stream 22 is received by one of two water adsorption systems 156 arranged in parallel. The drying system 120 may have two additional shutoff valves 158 to selectively cut off the flow of the fuel electrode output stream 22 to the water adsorption systems 156. During operation of the SOEC system 130, a first of the two shutoff valves 158 may be opened and the second shutoff valve 158 may be closed, allowing the fuel electrode output stream 22 to be supplied to a first of the two water absorption systems 156. Water adsorption systems 156 may each include a desiccant bed configured to adsorb water (water vapor) from the fuel electrode output stream 22. When the desiccant bed of the first water adsorption system 156 becomes saturated, the first shutoff valve 158 may be closed and the second shutoff valve 158 may be opened, allowing the fuel electrode output stream 22 to be supplied to the second water adsorption system 156. The desiccant bed of the first water adsorption system 156 may then be regenerated while the desiccant bed of the second water adsorption system 156 absorbs water from the fuel electrode output stream 22. The two water absorption systems 156 may alternate between receiving the fuel electrode output stream 22 and being regenerated such that the flow of the fuel electrode output stream 22 continues uninterrupted.

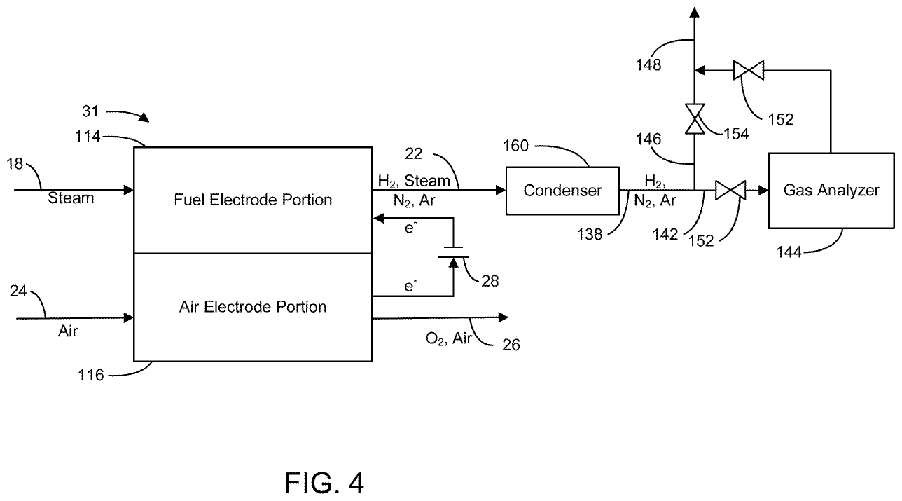

Referring now to FIG. 4, the SOEC system 130 is shown according to another exemplary embodiment. The SOEC system 130 of FIG. 4 may be substantially the same as the SOEC systems 130 of FIGS. 2 and 3 with the exception of the drying system 120. In the drying system 120 of FIG. 4, the fuel electrode output stream 22 is received by a condenser 160, which may cool the fuel electrode output stream 22 to a temperature at which substantially all of the water vapor condenses. As used herein, the phrase “substantially all” means that the remaining gas mixture contains less than 4.0% water vapor. The water vapor in the fuel electrode output stream 22 may condense, and the liquid water may be separated from the remaining gases in the fuel electrode output stream 22. It should be understood that any of the above drying systems 120 (e.g., in FIGS. 2-4) may be combined in series to dry the fuel electrode output stream 22. Thus, for example, the condenser 160 may remove less than substantially all of the water in the fuel electrode output stream 22, but substantially all of the remaining water may be removed in a membrane dryer 122 downstream of the condenser 160. In addition, drying systems 120 may include multiple drying components arranged in series. For example, the drying system 120 of FIG. 3 may include a second pair of water adsorption systems 156, or the drying system 120 of FIG. 4 may include a second condenser 160.

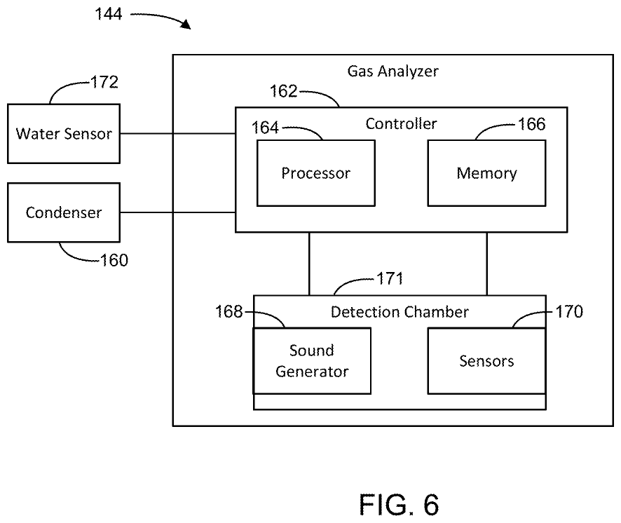

Referring now to FIG. 6, a schematic diagram of the binary gas analyzer 144 is shown according to an exemplary embodiment. The binary gas analyzer includes a controller 162 including at least one processor 164 and at least one memory 166. The at least one memory 166 may store instructions that may be executed by the at least one processor 164 to execute the functions of the controller 162. The binary gas analyzer 144 also includes a sound generator 168 and one or more sensors 170. The sensors 170 may include a sound sensor configured to detect sounds generated by the sound generator 168. The sensors 170 may also include a temperature sensor. The sound generator 168 and a sound sensor 170 may be positioned on opposite sides of a detection chamber 171 containing the gas to be analyzed (e.g., the first portion 142 of the drying system output stream 138). The controller 162 may send a signal to the sound generator 168 causing the sound generator 168 to generate a sound. The sound sensor 170 may detect the sound generated by the sound generator 168 and send a signal to the controller 162 indicating that the sound has been detected. The controller 162 may determine an amount of time between the sound being generated by the sound generator 168 and being detected by the sound sensor 170. Based on a known distance between the sound generator 168 and the sound sensor 170, the controller 162 may calculate the speed of a sound through a gas mixture in the binary gas analyzer 144.

Based on the determined speed of the sound through the gas mixture and a temperature of the gas (e.g., the first portion 142 of the drying system output stream 138) measured, for example, by a temperature sensor 170, the controller 162 may determine the hydrogen concentration in the gas. As discussed above, in a binary gas mixture, the concentrations of gas species in a binary gas mixture can be determined when the speed of a sound through the mixture is known. However, when the gas mixture contains a third gas species, the concentrations of the gases have not been determinable in typical binary gas analyzers. In the binary gas analyzer 144, the controller 162 is configured to determine the concentration of hydrogen in the drying system output stream 138.

Because the drying system output stream 138 is generated using the SOEC assembly 31, and because the drying system 120 is configured to remove substantially all of the water in the fuel electrode output stream 22, the only contaminants in drying system output stream 138 in any significant concentrations may be nitrogen and argon, with the remainder of the gas mixture being hydrogen. Any remaining trace gases, such as carbon dioxide, neon, helium, etc. that may be present in air and cross over the electrolytes 12 and mix into the fuel electrode output stream 22, may be present in such small concentrations (e.g., under 0.1%) as to be insignificant in the determination of the concentrations of nitrogen, hydrogen, and argon. Accordingly, the concentrations (c) of hydrogen, nitrogen, and argon may be assumed to add up to 100 percent of the gas mixture, as expressed in the formula:

c_{H}_{₂}+c_{N}_{₂}+c_{Ar}=1

It may be substantially certain that the nitrogen and argon in the drying system output stream 138 are present due to crossover from the air electrode 16, which receives the air in the air electrode input stream 24, and nitrogen and argon may cross over to the fuel electrode portion 114 at similar rates. Thus, it may be substantially certain that the nitrogen and argon in the drying system output stream 138 are present in substantially the same ratio as they are present in air. This ratio is approximately 78:1 nitrogen to argon. Thus, the concentration of argon in the drying system output stream 138 may be expressed as

$${c}_{Ar} = \frac{{c}_{{N}_{2}}}{78}$$

The concentration of argon in the formula above may be replaced with this expression, yielding a formula of:

$${c}_{{H}_{2}} + {c}_{{N}_{2}} + \frac{{c}_{{N}_{2}}}{78} = 1 or {c}_{{H}_{2}} + \frac{79}{78} \cdot {c}_{{N}_{2}} = 1$$

and the concentration of nitrogen may be expressed as:

$${c}_{{N}_{2}} = \frac{78}{79} ( 1 - {c}_{{H}_{2}} )$$

As discussed above, the speed of a sound in through a gas mixture can be determined using the formula (referred to herein as the “Sound Speed Formula”):

$$S ( mix ) = \sqrt{\frac{{γ}_{mix} RT}{{M}_{mix}}}$$

where Mₘᵢₓ=ΣcᵢMᵢ,

$${γ}_{mix} = 1 + \frac{R}{\sum {c}_{i} {M}_{i} {C}_{{V}_{i}}} ,$$

T is the temperature of the gas, M is the molar mass of a particular gas species, c is the percentage of a particular gas species in the mixture, R is the ideal gas constant, and C_{V} is the specific heat of a particular gas species. Because the binary gas analyzer 144 is configured to determine the speed of a sound through a gas mixture, and because the concentrations of argon and nitrogen can be expressed as functions of the concentration of hydrogen, the concentration of hydrogen may be the only unknown in the equations above, and the controller 162 may determine the concentration of hydrogen based on the speed of a sound through the gas mixture (e.g., the drying system output stream 138). Thus, the binary gas analyzer 144 is configured to determine the concentration of hydrogen in the drying system output stream 138 containing more than two gas species.

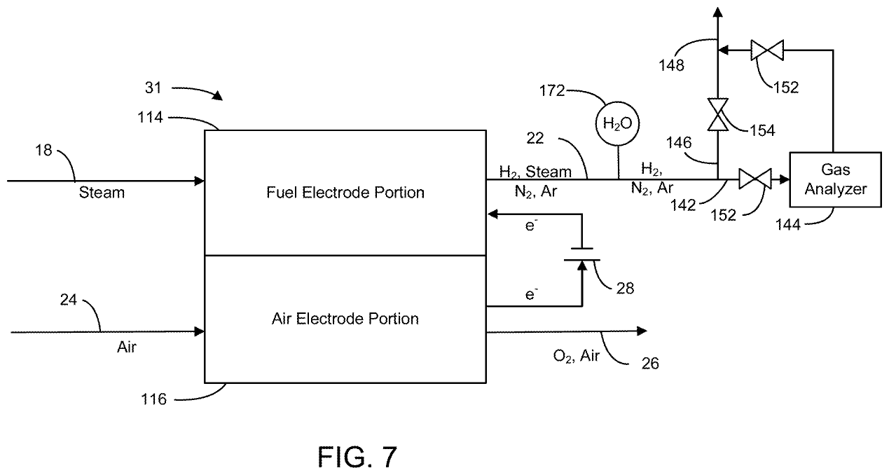

Referring now to FIG. 7, the SOEC system 130 is shown according to another exemplary embodiment. The SOEC system 130 of FIG. 7 may be substantially the same as the SOEC system 130 of FIGS. 2-4 except that the SOEC system 130 of FIG. 7 does not include a drying system 120. Instead, the SOEC system 130 of FIG. 7 includes a water concentration sensor 172 upstream of the binary gas analyzer 144. The water concentration sensor 172 is configured to receive and determine the water concentration of the fuel electrode output stream 22. In some embodiments, the SOEC system 130 may include both a drying system 120 as shown in FIGS. 2-4 and a water concentration sensor 172. For example, the drying system 120 may only partially dry the fuel electrode output stream 22 rather than removing substantially all of the water vapor from the fuel electrode output stream 22. The water concentration sensor 172 may then determine the concentration of water vapor in the partially-dried fuel electrode output stream 22 (drying system output stream 138). Because the water vapor (or not all of the water vapor) is not removed from the fuel electrode output stream 22, the concentrations (c) of hydrogen, nitrogen, argon, and water vapor may add up to 100 percent of the gas mixture, as expressed in the formula:

c_{H}_{₂}+c_{N}_{₂}+c_{Ar}+c_{H}_{₂}=1

The concentrations of nitrogen and argon can be replaced with the expression 78/79 (1−c_{H}_{₂}) as discussed above, and the concentration of water vapor (H₂O) can be determined based on the equilibrium temperature at which liquid water was separated from the fuel electrode output stream 22 Thus, the formulas above for determining the speed of a sound through a gas mixture can be used to determine the concentration of hydrogen in the mixture, as the hydrogen concentration is the only unknown in the equations.

As discussed above, the speed of a sound through a gas mixture can be determined using the Sound Speed Formula:

$$S ( mix ) = \sqrt{\frac{{γ}_{mix} RT}{{M}_{mix}}}$$

where Mₘᵢₓ=ΣcᵢMᵢ,

$${γ}_{mix} = 1 + \frac{R}{\sum {c}_{i} {M}_{i} {C}_{{V}_{i}}} ,$$

T is the temperature of the gas, M is the molar mass of a particular gas species, c is the percentage of a particular gas species in the mixture, R is the ideal gas constant, and C_{V} is the specific heat of a particular gas species. Because the binary gas analyzer 144 is configured to determine the speed of a sound through a gas mixture, and because the concentrations of argon and nitrogen can be expressed as functions of the concentration of hydrogen, the concentration of hydrogen may be the only unknown in the equations above, and the controller 162 may determine the concentration of hydrogen based on the speed of a sound through the gas mixture (e.g., the drying system output stream 138). Thus, the binary gas analyzer 144 is configured to determine the concentration of hydrogen in the drying system output stream 138 containing more than two gas species.

Referring again to FIG. 4, in some embodiments, the SOEC system 130 may include a condenser 160 that is configured to only partially remove water vapor from the fuel electrode output stream 22 but may not include a water concentration sensor 172. The condenser 160 may be configured to cool the fuel electrode output stream 22 to a desired temperature to condense some of the water to form a two-phase mixture at thermal equilibrium. Based on the temperature to which the two-phase mixture is cooled, the concentration of water vapor in the drying system output stream 138 can be determined approximately (but to a precision sufficient for practical needs) using, for example, various thermodynamic models. Thus, the water concentration sensor 172 may not be needed to determine the concentration of water vapor in the drying system output stream 138 or to subsequently determine the concentration of hydrogen. The calculation of the concentration of hydrogen can be performed as discussed above, using the water vapor concentration calculated based on the temperature at which the drying system output stream 138 was separated from the two-phase mixture.

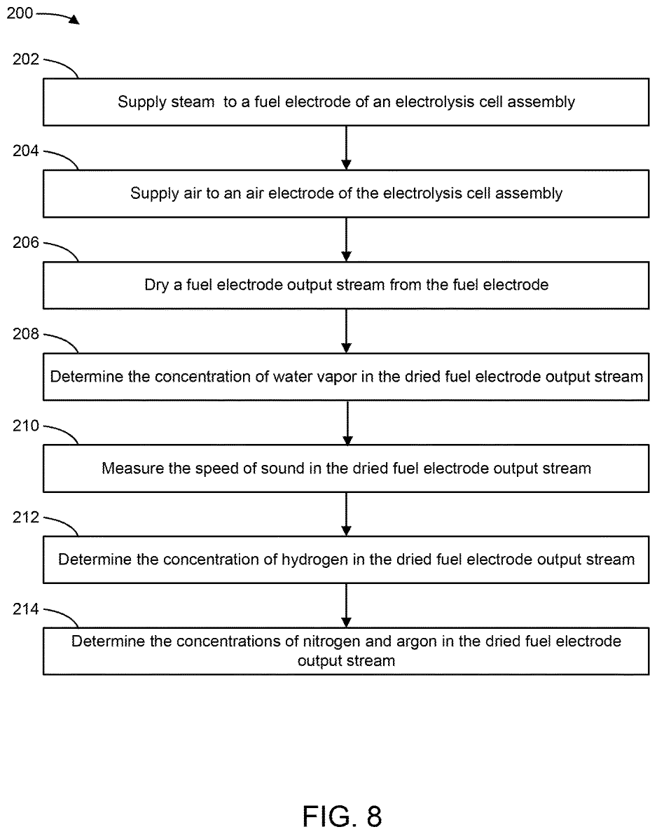

Referring now to FIG. 8, a method 200 of measuring the concentration of hydrogen in a gas mixture generated by an electrolysis system is shown, according to an exemplary embodiment. The method may be performed, for example, using the SOEC system 130 of FIG. 2-6 or 7 including the gas analyzer 144. At operation 202 of the method 200, fuel is supplied to a fuel electrode of an electrolysis cell assembly, and at operation 204 of the method 200, air is supplied to an air electrode of the electrolysis cell assembly. The electrolysis cell assembly may be, for example, a solid oxide electrolysis cell assembly (e.g., the SOEC assembly 31).

At operation 206 of the method 200, a fuel electrode output stream from the fuel electrode of the electrolysis cell assembly is dried. The fuel electrode output stream may consist substantially of hydrogen, nitrogen, argon, and water vapor. In some embodiments, the method 200 may not include operation 206 and the fuel electrode output stream may not be dried. Drying the fuel electrode output stream may include, for example, passing the fuel electrode output stream through a membrane dryer (e.g., the membrane dryer 122). The fuel electrode output stream may be supplied to a retentate side of the membrane dryer, and a sweep gas stream including hydrogen or nitrogen may be supplied to a permeate side of the membrane dryer. In some embodiments, drying the fuel electrode output stream may include passing the fuel electrode output stream through an adsorption drying system (e.g., the water adsorption system 156). In some embodiments, drying the fuel electrode output stream may include cooling the fuel electrode output stream to condense water vapor present in the fuel electrode output stream in a condenser (e.g., the condenser 160).

At operation 208 of the method 200, the concentration of water vapor in the dried fuel electrode output stream is determined. In some embodiments, a water concentration sensor (e.g., the water concentration sensor 172) may be used to determine the concentration of water vapor in the dried fuel electrode output stream. In embodiments of the method 200 that do not include operation 206, operation 208 may include measuring the water concentration of the fuel electrode output stream without previously drying the fuel electrode output stream. In some embodiments, the concentration of water vapor in the dried fuel electrode output stream may be determined based on the temperature to which the fuel electrode output stream was cooled in the condenser. By separating the dried fuel electrode output stream from a two-phase mixture at a known temperature at thermal equilibrium, the concentration of water in the dried fuel electrode output stream can be determined.

At operation 210 of the method 200, the speed of a sound through the dried fuel electrode output stream (or the undried fuel electrode output stream) is measured. For example, the dried fuel electrode output stream may be input into a chamber of a gas analyzer (e.g., gas analyzer 144) including a sound generator and a sound sensor, and the amount of time between the sound generator generating a sound and the sound being detected by the sound sensor may be used to determine the speed of a sound through the dried fuel electrode output stream. In some embodiments, other methods, such as the pulse-echo method performed with an ultrasonic sensor, may be used to measure the speed of a sound through the gas mixture.

At operation 212 of the method 200, the concentration of hydrogen in the dried fuel electrode output stream is determined based on the speed of a sound through the dried fuel electrode output stream and the determined concentration of water vapor in the dried fuel electrode output stream using the formulas discussed above. As discussed above, the concentration of argon may be assumed to be approximately 1/78 the concentration of nitrogen, approximately the same ratio present in atmospheric air. At operation 214 of the method 200, the concentrations of nitrogen and argon in the dried fuel electrode output stream are determined based on the determined concentrations of water vapor and hydrogen in the dried fuel electrode output stream. Again, the concentration of argon may be assumed to be approximately 1/78 the concentration of nitrogen when determining the concentrations of nitrogen and argon.

While the embodiments described herein relate specifically to measuring the concentration of gases output by the fuel electrode portion of an electrolysis cell system, it should be understood that similar methods may be used to determine the concentrations of gases in gas mixtures produced by other methods. For example, the SOEC assembly 31 may be operated in reverse as a fuel cell assembly by supplying fuel, such as hydrogen, to the fuel electrode portion 114 and air to the air electrode portion 116. The fuel cell assembly may generate an electric current from the fuel, and the hydrogen may react with the oxide ions that cross over the electrolytes 12 of the fuel cells from the air electrodes 16 to the fuel electrodes 14 to form water vapor. Nitrogen and argon may also cross over the electrolytes 12 as discussed above. The fuel electrode may output a fuel electrode output stream consisting substantially of hydrogen, water vapor, nitrogen, and argon, similar to the fuel electrode output stream in the SOEC assembly 31 operated in electrolysis mode, though potentially in different proportions. The hydrogen concentration of the fuel electrode output stream from the fuel cell assembly can then be analyzed in the same way as the fuel electrode output stream from the SOEC assembly 31.

Similar methods may be used in other cases where a correlation between two non-condensable gases can be determined, similar to the correlation between nitrogen and argon discussed above, or when the concentration of a condensable gas can be determined independently as the concentration of water is determined above. For example, a gas mixture consisting substantially of a condensable vapor, a first gas species, a second gas species, and a third gas species, where the second gas species and the third gas species are in a known ratio, may be analyzed to determine the concentrations of the gas species and condensable vapor in the gas mixture. At least some of the condensable vapor may be condensed to a liquid, leaving a residual gas mixture. The concentration of the condensable vapor in the gas mixture may then be determined using the methods discussed above with respect to water vapor. For example, the gas mixture may be cooled to form a two-phase mixture, and based on the temperature to which the two-phase mixture is cooled, the concentration of the condensable vapor in the residual gas mixture can be determined. Alternatively, the condensable vapor may be completely condensed and removed from the gas mixture. Next the speed of a sound through the residual gas mixture may be measured, and the concentration of the first gas species in the residual gas mixture may be calculated as a function of the speed of the sound (e.g., using the Sound Speed Formula) through the residual gas mixture with the concentrations of the second gas species and the third gas species are presumed to be in the known ratio. As used herein a “condensable” gas or vapor refers to a pure substance that can be condensed into a liquid by cooling to not lower than negative 40 degrees Celsius.

For example, over their evolution, landfills form considerable amounts of carbon dioxide and methane. Methane is of economic value and is often extracted, separated from carbon dioxide and other impurities, and either compressed and sold, converted to electricity, or burned to provide heat. Other gases such as oxygen, hydrogen, and nitrogen are present in percentages that depend on the age of the landfill. Their typical percentage can be correlated to the age of the landfill or the relative percentage of carbon dioxide and methane. Using these known correlations, the percentages of more than two gases can be determined from the approximate carbon dioxide/methane concentration.

In some embodiments, the residual gas mixture may be provided to a binary gas analyzer and the speed of a sound through the residual gas mixture may be determined, but the binary gas analyzer may output estimated concentrations of two of the gas species rather than outputting the measured speed of sound. For example, for a gas mixture containing hydrogen, nitrogen and argon (with nitrogen and argon being in a known ratio) the binary gas analyzer may be configured to output estimated concentrations of hydrogen and nitrogen in a gas mixture, which are assumed in the calculation by the binary gas analyzer to be the only gas species in the gas mixture, but may not be configured to output the speed of sound used to determine those concentrations. In some embodiments, a method of analyzing a gas mixture containing more than two non-condensable gas species may include determining the speed of the sound measured by the binary gas analyzer. For example, using the Sound Speed Formula with the estimated concentrations of hydrogen and nitrogen being known and with the assumption that the gas mixture consists only of hydrogen and nitrogen (as is the assumption used by the binary gas analyzer), the speed of the sound measured by the binary gas analyzer can be determined. With the speed of the sound known, the concentrations of hydrogen, nitrogen, and argon (e.g., the “actual” concentrations, rather than the estimated concentrations form the binary gas analyzer) may be calculated using the Sound Speed Formula as discussed above.

As utilized herein with respect to numerical ranges and values, the terms “approximately,” “about,” “substantially,” and similar terms generally mean+/−10% of the disclosed values, unless specified otherwise. As utilized herein with respect to structural features (e.g., to describe shape, size, orientation, direction, relative position, etc.), the terms “approximately,” “about,” “substantially,” and similar terms are meant to cover minor variations in structure that may result from, for example, the manufacturing or assembly process and are intended to have a broad meaning in harmony with the common and accepted usage by those of ordinary skill in the art to which the subject matter of this disclosure pertains. Accordingly, these terms should be interpreted as indicating that insubstantial or inconsequential modifications or alterations of the subject matter described and claimed are considered to be within the scope of the disclosure as recited in the appended claims.

As utilized herein, the phrase “consisting substantially of” referring to the content of a gas mixture means that the listed contents make up at least 95.0 percent of the total volume of the gas mixture. Thus, a gas mixture consisting substantially of hydrogen, nitrogen, argon, and water vapor contains at least 95.0 percent hydrogen, nitrogen, argon, and water vapor and less than 5.0 percent other gas species. In some embodiments, a gas mixture may be described as “consisting of at least” one or more species as a percentage of the gas mixture. For example, a gas mixture “consisting of at least 98.0 percent” hydrogen, nitrogen, argon, and water vapor contains at least 98.0 percent hydrogen, nitrogen, argon, and water vapor and less than 2.0 percent other gas species.

The terms “coupled,” “connected,” and the like as used herein mean the joining of two members directly or indirectly to one another. Such joining may be stationary (e.g., permanent) or moveable (e.g., removable or releasable). Such joining may be achieved with the two members or the two members and any additional intermediate members being integrally formed as a single unitary body with one another or with the two members or the two members and any additional intermediate members being attached to one another.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The hardware and data processing components used to implement the various processes, operations, illustrative logics, logical blocks, modules and circuits described in connection with the embodiments disclosed herein may be implemented or performed with a general-purpose single- or multi-chip processor, a digital signal processor (DSP), an application specific integrated circuit (ASIC), a field programmable gate array (FPGA), or other programmable logic device, discrete gate or transistor logic, discrete hardware components, or any combination thereof designed to perform the functions described herein. A general-purpose processor may be a microprocessor, or, any conventional processor, controller, microcontroller, or state machine. A processor also may be implemented as a combination of computing devices, such as a combination of a DSP and a microprocessor, a plurality of microprocessors, one or more microprocessors in conjunction with a DSP core, or any other such configuration. In some embodiments, particular processes and methods may be performed by circuitry that is specific to a given function. The memory (e.g., memory, memory unit, storage device) may include one or more devices (e.g., RAM, ROM, Flash memory, hard disk storage) for storing data and/or computer code for completing or facilitating the various processes, layers and modules described in the present disclosure. The memory may be or include volatile memory or non-volatile memory, and may include database components, object code components, script components, or any other type of information structure for supporting the various activities and information structures described in the present disclosure. According to an exemplary embodiment, the memory is communicably connected to the processor via a processing circuit and includes computer code for executing (e.g., by the processing circuit or the processor) the one or more processes described herein.

The present disclosure contemplates methods, systems and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general purpose computer, special purpose computer, or special purpose processing machines to perform a certain function or group of functions.

What is claimed is:

1. A hydrogen production system comprising:
an electrolysis cell system comprising:
a fuel electrode portion configured to receive a fuel electrode input stream comprising gaseous fuel and to output a fuel electrode output stream consisting substantially of hydrogen, nitrogen, argon, and water vapor; and
an air electrode portion configured to receive an air electrode input stream comprising air;
a drying system configured to dry the fuel electrode output stream; and
a gas analyzer configured to receive a first portion of the dried fuel electrode output stream, the gas analyzer comprising a sound generator, a sound sensor, and a controller, the controller comprising at least one processor and at least one memory storing instructions that, when executed by the at least one processor, cause the controller to:
cause the sound generator to generate a sound;
determine, based on a signal from the sound sensor, the speed of the sound through the first portion of the dried fuel electrode output stream; and
calculate the concentration of hydrogen in the first portion of the dried fuel electrode output stream as a function of the speed of the sound through the first portion of the dried fuel electrode output stream, wherein the concentration of argon is assumed to be approximately 1/78 the concentration of nitrogen.

2. The hydrogen production system of claim 1, wherein the concentration of hydrogen is calculated using the formula:
$$S ( mix ) = \sqrt{\frac{{γ}_{mix} RT}{{M}_{mix}}}$$
where Mₘᵢₓ=ΣcᵢMᵢ,
$${γ}_{mix} = 1 + \frac{R}{\sum {c}_{i} {M}_{i} {C}_{{V}_{i}}} ,$$
T is the temperature of the first portion, S is the speed of the sound through the first portion, M is the molar mass of a particular gas species, c is the percentage of a particular gas species, R is the ideal gas constant, C_{V} is the specific heat of a particular gas species.

3. The hydrogen production system of claim 1, wherein the drying system is configured to remove substantially all of the water in the fuel electrode output stream.

4. The hydrogen production system of claim 1, wherein the drying system comprises at least one of a condenser, a membrane dryer, or an adsorption system.

5. The hydrogen production system of claim 1, wherein the drying system comprises a membrane dryer comprising a retentate portion configured to receive the fuel electrode output stream, a permeate portion configured to receive a sweep gas stream comprising one of nitrogen or hydrogen, and a membrane separating the retentate portion and the permeate portion.

6. The hydrogen production system of claim 1, further comprising a water concentration sensor configured to measure the concentration of water vapor in the dried fuel electrode output stream, wherein the controller is further configured to receive the measured concentration of water vapor, and the calculation of the concentration of hydrogen is further based on the measured concentration of water vapor.

7. The hydrogen production system of claim 1, wherein the drying system comprises a condenser configured to cool the fuel electrode output stream to a first temperature at thermal equilibrium, wherein the controller is further configured to receive the first temperature and determine, based on the first temperature, the concentration of water vapor in the dried fuel electrode output stream, wherein the calculation of the concentration of hydrogen is further based on the calculated concentration of water vapor.

8. The hydrogen production system of claim 1, wherein the electrolysis cell system includes a plurality of solid oxide electrolysis cells.

9. A method of measuring the concentration of hydrogen in a gas mixture, the method comprising:
supplying a steam stream to a fuel electrode portion of an electrolysis cell system;
supplying an air stream to an air electrode portion of the electrolysis cell system;
drying a fuel electrode output stream output from the fuel electrode portion, the fuel electrode output stream consisting substantially of hydrogen, nitrogen, argon, and water vapor;
measuring the speed of a sound in a first portion of the dried fuel electrode output stream; and
calculating the concentration of hydrogen in the first portion of the dried fuel electrode output stream as a function of the speed of the sound through the first portion of the dried fuel electrode output stream, wherein the concentration of argon is assumed to be approximately 1/78 the concentration of nitrogen.

10. The method of claim 9, wherein the concentration of hydrogen in the first portion of the dried fuel electrode output stream is calculated using the formula:
$$S ( mix ) = \sqrt{\frac{{γ}_{mix} RT}{{M}_{mix}}}$$
where Mₘᵢₓ=ΣcᵢMᵢ,
$${γ}_{mix} = 1 + \frac{R}{\sum {c}_{i} {M}_{i} {C}_{{V}_{i}}} ,$$
T is the temperature of the first portion, S is the speed of the sound through the first portion, M is the molar mass of a particular gas species, c is the percentage of a particular gas species, R is the ideal gas constant, C_{V} is the specific heat of a particular gas species, and the concentration of argon is assumed to be approximately 1/78 the concentration of nitrogen.

11. The method of claim 9, wherein drying the fuel electrode output stream comprises removing substantially all of the water in the fuel electrode output stream.

12. The method of claim 9, wherein drying the fuel electrode output stream comprises supplying the fuel electrode output stream to a retentate portion of a membrane dryer and supplying one of a hydrogen stream or a nitrogen stream to a permeate portion of the membrane dryer.

13. The method of claim 9, further comprising measuring the concentration of water vapor in the dried fuel electrode output stream, wherein the calculation of the concentration of hydrogen in the first portion of the dried fuel electrode output stream is further based on the measured concentration of water.

14. The method of claim 9, wherein drying the fuel electrode output stream comprises cooling the fuel electrode output stream to a first temperature at thermal equilibrium, wherein the method further comprises determining the concentration of water in the dried fuel electrode output stream based on the first temperature.

15. The method of claim 14, wherein the calculation of the concentration of hydrogen in the first portion of the dried fuel electrode output stream is further based on the determined concentration of water in the dried fuel electrode output stream.

16. The method of claim 9, further comprising determining the concentrations of nitrogen and argon in the dried fuel electrode output stream.

17. A gas analyzer comprising:
a detection chamber;
a sound generator positioned on a first side of the detection chamber;
a sound sensor positioned on a second side of the detection chamber; and
a controller comprising at least one processor and at least one memory storing instructions that, when executed by the at least one processor, cause the controller to:
cause the sound generator to generate a sound;
determine, based on a signal from the sound sensor, the speed of the sound through a gas mixture in the detection chamber, the gas mixture consisting substantially of hydrogen, nitrogen, and argon, or hydrogen, nitrogen, argon, and water vapor; and
calculate the concentration of hydrogen in the gas mixture as a function of the speed of the sound through the gas mixture, wherein the concentration of argon is assumed to be approximately 1/78 the concentration of nitrogen.

18. The gas analyzer of claim 17, wherein the concentration of hydrogen in the gas mixture is calculated using the formula:
$$S ( mix ) = \sqrt{\frac{{γ}_{mix} RT}{{M}_{mix}}}$$
where Mₘᵢₓ=ΣcᵢMᵢ,
$${γ}_{mix} = 1 + \frac{R}{\sum {c}_{i} {M}_{i} {C}_{{V}_{i}}} ,$$
T is the temperature of the gas mixture, S is the speed of the sound through the gas mixture, M is the molar mass of a particular gas species, c is the percentage of a particular gas species, R is the ideal gas constant, C_{V} is the specific heat of a particular gas species, and the concentration of argon is assumed to be approximately 1/78 the concentration of nitrogen.

19. The gas analyzer of claim 17, wherein the instructions further cause the controller to receive a measured concentration of water vapor of the gas mixture, wherein the calculation of the concentration of hydrogen is further based on the measured concentration of water vapor.

20. The gas analyzer of claim 17, wherein the instructions further cause the controller to receive a first temperature to which a condenser cools and condenses water out of the gas mixture at thermal equilibrium before the speed of the sound is measured, wherein the calculation of the concentration of hydrogen is further based on the first temperature.
